(19) United States
(12) Patent Application Publication
(10) Pub. No.: US 2005/0100618 A1
(43) Pub. Date: May 12, 2005

(54) Method of using a compound of menthol and L-arginine as a preparation for the topical delivery of Icariin, a herbal product produced from the Epimedium genus of the Berberidaceal family of plants, for the treatment of sexual dysfunction

(76) Inventors: Ronald J. Thompson, Ft. Thomas, KY (US); James M. Thompson, Cincinnati, OH (US); Jonathan R. Thompson, Cincinnati, OH (US); Justin R. Thompson, Cincinnati, OH (US); Robert C. Hassman, Fort Thomas, KY (US); Robert M. Williams, Fort Thomas, KY (US)

Correspondence Address: Donald N. Halgren, 35 Central Street, Manchester, MA, 01944, US

(21) Appl. No.: 11/014,429

(22) Filed: Dec. 16, 2004

Related U.S. Application Data
(63) Continuation-in-part of application No. 10/731,692, filed on Dec. 9, 2003.
Application No. 10/731,692 is a division of application No. 10/004,091, filed on Oct. 23, 2001, now Pat. No. 6,702,733.
Application No. 10/004,091 is a continuation of application No. 09/520,110, filed on Mar. 7, 2000, now Pat. No. 6,322,493.
Application No. 09/520,110 is a continuation-in-part of application No. 09/469,959, filed on Dec. 21, 1999, now abandoned.
Application No. 09/469,959 is a continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61K 35/78; A61K 31/198; A61K 31/045

(52) U.S. Cl. 424/739; 424/747; 424/742; 514/565; 514/729

(57) ABSTRACT

A manual application of a topical genital sensitizing compound combination consisting of L-arginine and a cooling agent comprised of Menthol and Icariin, a herbal product produced from the Epimedium genius of the Berberidaceal family of plants, wherein the combination is applicable manually to the genitalia.

### BACKGROUND OF THE INVENTION

1. Field of the Invention

This invention relates to arrangements for the sexual stimulation through the topical application of specialized stimulatory medicaments. This application is a continuation-in-part application of co-pending U.S. patent application Ser. No. 10/731,692 filed 9 Dec. 2003 which is a divisional application of U.S. patent application Ser. No. 10/004,091, filed 23 Oct. 2001, now U.S. Pat. No. 6,702,733 issued 9 Mar. 2004, which is a continuation of application Ser. No. 09/520,110, filed 7 Mar. 2000, now U.S. Pat. No. 6,322,493 which is a continuation-in-part of application Ser. No. 09/469,959 filed on 21 Dec. 1999, which is a continuation-in-part of application Ser. No. 09/414,250, filed on 7 Oct. 1999, now Pat. No. 6,224,541 which is a continuation-in-part of application Ser. No. 09/340,227, filed on 1 Jul. 1999, now U.S. Pat. No. 6,179,775 each of which are incorporated herein by reference in their entirety.

2. Prior Art

In U.S. Pat. Nos. 6,322,493 and 6,702,733 we describe a combination of menthol and L-arginine, topically applied to the mucous membrane of the female clitoris, to increase vasodilatation of the corpus cavemosa. The menthol initially causes vasodilatation and secondarily functions as a lipophilic vehicle to enhance the clitoral absorption of L-arginine. The increased tissue concentrations of L-arginine cause induction of the nitric oxide synthase pathway, production of nitric oxide and cyclic-GMP, for the persistent vasodilatation of the corpus cavemosa.

Patent application Ser. No. 10/803,148, filed Mar. 17, 2004, incorporated herein by reference, entitled: “A method of using a compound of menthol and L-arginine as a preparation for the topical delivery of a 5-phosphodiesterase Inhibitor for the Treatment of female sexual dysfunction” takes this treatment further. The invention recited therein dercribes a treatment method whereby using the menthol component of our compound also as a vehicle for the enhanced topical absorption of a naturally occurring 5-phospohdiesterase inhibitor, Icariin, an herbal product produced from the Epimedium genus of the Berberidoecae family of plants.

The present invention relates to the substitution of a naturally occurring 5PDE-Icariin, produced from the Epimedium genus of plants, for the 5-phosphodiesterase inhibitor, in combination with topically applied menthol and L-arginine for the treatment of sexual dysfunction.

The combination of three vasodilators, menthol, L-arginine, and Icariin, all with different methods of action for inducing vasodilatation allows for reducing the dosage of each and still achieving effectiveness of corpus cavernosa enlargement and sensitivity and rigidity. The menthol component of our proposed combination would also act as a vehicle for the absorption of the L-arginine and Icariin. Recent medical literature reports have identified and confirmed a naturally occurring 5 Phosphodiesterase inhibitor. In the Asian Journal of Andrology, March 2003, Dr Xin et al report “Effects of icarim on cGMP-specific PDE-5 and cAMP-specific PDE-5 activities” that Icariin is a specific cGMP-specific inhibitor that may be developed into an oral effective agent for the treatment of Erectile Dysfunction.

This inventive disclosure describes the topical use of the combination of menthol, L-arginine, and Icariin, a naturally occurring 5PDE-I for the topical treatment of sexual dysfunction. Therefore, the previously un-described combination of menthol/L-arginine and Icariin presents a multitude of synergistic interactions between and amongst the individual components that would be much more effective in the treatment of sexual dysfunction, and at much lower concentrations of each of the components.

### DETAILED DESCRIPTION OF THE PRESENT INVENTION

The present invention relates to a manually applicable topical preparation comprising a compound of menthol or a menthol substitute, and L-arginine, and Icariin as that topical compound. This manually applicable compound is to be manually applied by an applicator directly to the genitalia for the treatment of sexual dysfunction. Such Icariin in one preferred embodiment may preferably comprise less than 20% of that topical compound.

A first preferred embodiment of a Menthol substitute may be peppermint oil. A second preferred embodiment of a Menthol substitute may be Cornmint oil. A third preferred embodiment of a Menthol substitute may be Eucalyptus oil. A fourth preferred embodiment of a Menthol substitute may be Citronella oil. A fifth preferred embodiment of a Menthol substitute may be Camphor oil. A sixth preferred embodiment of a Menthol substitute may be Cinnamon oil. A seventh preferred embodiment of Menthol would include Menthol analogs and derivatives including: (+)-neo-Menthol, Menthone, (+)-iso-Menthone, Menthyl acetate, Menthyl isovalerate, (−)-Menthyl lactate, para-Menth-1-en-3-ol, Piperitone, (−)-Menthol ethylene glycol carbonate, (−)Menthol 1- and 2-propylene glycol carbonate, (−)-Mentholel 1,2-glycerol ketal, (+)-Menthonel 1,2-glycerol ketal, and mono-Methyl succinate.

The invention thus comprises a manual application of a topical sensitizing combination consisting essentially of L-arginine and a cooling agent comprised of Menthol and Icarin, wherein the combination is applicable manually. The combination in one preferred embodiment is contained in a hand manipulable reservoir, an applicator or a container to facilitate direct application to, or to the hand of the person for such hand application.

The cooling agent may in various preferred embodiments include peppermint oil. The cooling agent may include cornmint oil. The cooling agent may include Eucalypus oil. The cooling agent may include Citronella oil. The cooling agent may include Camphor oil. The cooling agent may include Cinnamon oil. The cooling agent may essentially comprise peppermint oil. The cooling agent may essentially comprise cornmint oil. The cooling agent may essentially comprise Eucalyptus oil. The cooling agent may essentially comprise Citronella oil. The cooling agent may essentially comprise Camphor oil. The cooling agent essentially may comprise Cinnamon oil. The combination may contain less than 10% L-arginine. The combination may contain less than 10% menthol and the combination may contain less than 10% L-arginine and also the combination may contain less than 20% Icariin.

1. A manual application of a topical sexual sensitizing combination consisting essentially of L-arginine and a cooling agent comprised of Menthol and Icarin, wherein said combination is applicable manually to the clitoris.

2. The topical sensitizing combination as recited in claim 1, wherein said cooling agent includes peppermint oil.

3. The topical sensitizing combination as recited in claim 1, wherein said cooling agent includes cornmint oil.

4. The topical sensitizing combination as recited in claim 1, wherein said cooling agent includes Eucalypus oil.

5. The topical sensitizing combination as recited in claim 1, wherein said cooling agent includes Citronella oil.

6. The topical sensitizing combination as recited in claim 1, wherein said cooling agent includes Camphor oil.

7. The topical sensitizing combination as recited in claim 1, wherein said cooling agent includes Cinnamon oil.

8. The topical sensitizing combination as recited in claim 1, wherein said cooling agent essentially comprises peppermint oil.

9. The topical sensitizing combination as recited in claim 1, wherein said cooling agent essentially comprises cornmint oil.

10. The topical sensitizing combination as recited in claim 1, wherein said cooling agent essentially comprises Eucalyptus oil.

11. The topical sensitizing combination as recited in claim 1, wherein said cooling agent essentially comprises Citronella oil.

12. The topical sensitizing combination as recited in claim 1, wherein said cooling agent essentially comprises Camphor oil.

13. The topical sensitizing combination as recited in claim 1, wherein said cooling agent essentially comprises Cinnamon oil.

14. The topical sensitizing combination as recited in claim 1, wherein said combination contains less than 10% L-arginine.

15. The topical sensitizing combination as recited in claim 1, wherein said combination contains less than 10% menthol and said combination contains less than 10% L-arginine and said combination contains less than 20% Icariin.

16. A manual application of a topical sexual sensitizing combination consisting essentially of L-arginine and a cooling agent comprised of Menthol and Icariin, wherein said combination is applicable manually to the clitoris and said Menthol includes one or more analogs thereof selected from the group comprising:
a. (+)-neo-Menthol
b. Menthone
c. (+)-iso-Menthone
d. Menthyl acetate
e. Menthyl isovalerate
f. (−)-Menthyl lactate
g. para-menth-1-en-3ol
h. Piperitone
i. (−)-Menthol ethylene glycol carbonate
j. (−)-Menthol 1-and 2-propylene glycol carbonate
k. (−)-Menthone 1,2-glycerol ketal
l. (+)-Menthone 1,2-glycerol ketal
m. mono-Menthyl succinate.

17. A manual application of a topical sexual sensitizing combination consisting essentially of L-arginine and a cooling agent comprised of Menthol and Icariin, wherein said combination is applicable manually from an applicator to a human genital region.
